(19) United States
(12) Patent Application Publication
(10) Pub. No.: US 2011/0021845 A1
(43) Pub. Date: Jan. 27, 2011

(54) PREPARATION OF ALCOHOLS FROM ALDEHYDES

(76) Inventors: Danilo Zim, Campinas (BR); Philippe Marion, Vernaison (FR)

Correspondence Address: BUCHANAN, INGERSOLL & ROONEY PC, POST OFFICE BOX 1404, ALEXANDRIA, VA, 22313-1404, US

(21) Appl. No.: 12/747,647

(22) PCT Filed: Dec. 10, 2008
(86) PCT No.: PCT/IB08/03407

§ 371 Date: Sep. 1, 2010

(30) Foreign Application Priority Data
Dec. 14, 2007 (FR) 0708723

(51) Int. Cl. C07C 45/45; C07C 29/38

(52) U.S. Cl. 568/463; 568/881

(57) ABSTRACT

Alcohols are prepared from aldehydes employing a bifunctional catalyst; the aldehydes are first condensed and the products of condensation thereof are then converted into alcohols by hydrogenation.

The present invention relates to a process for obtaining alcohol from an aldehyde. More specifically, the present invention relates to a process for obtaining alcohol from an aldehyde using a bifunctional catalyst. It also relates to a process for the condensation of an aldehyde, to a process for obtaining alcohol on the basis of the hydrogenation of the product of condensation of an aldehyde, and also to the products obtained through each respective process.

### PRIOR ART

Butanol (1-butanol) is currently synthesized in two steps through the hydroformylation (carbonylation) of propene by means of a process known as oxo synthesis, producing butyraldehyde. The butyraldehyde is then hydrogenated, so as to obtain 1-butanol. The production of 1-butanol based on the hydroformylation of propene is dependent on the supply of this starting material originating from petroleum. Owing to the increased scarcity of this nonrenewable source and the gradual increase in the price thereof, the cost of 1-butanol production can make its use prohibitive. In addition, there is strong pressure around the world for petroleum to be replaced, as a source of starting material, by a resource derived from biomass, owing to the environmental impact that it causes.

Another process comprises aldol condensation of acetaldehyde followed by dehydration producing crotonaldehyde using sodium hydroxide as catalyst, followed by hydrogenation, producing 1-butanol. In this process, the yield is low and the crotonaldehyde is toxic, irritant and difficult to handle, and readily undergoes a polymerization reaction.

### INVENTION

The subject of the present invention is a process for obtaining alcohol from an aldehyde, comprising, in a first step, condensation of the aldehyde by dehydration, in the presence of a specific solid catalyst, forming a condensed aldehyde, followed by hydrogenation of the latter, producing alcohol, in a second step.

This process makes it possible to obtain an alcohol with excellent conversion and selectivity.

### First Step

The first step of this process is a process of condensation of two molecules of aldehyde in the presence of a catalytic system comprising an acidic or basic solid compound and of a metal compound, at a temperature of between 10° C. and 300° C. and a pressure of between 0.01 and 200 bar, in the presence of hydrogen.

The catalytic system may be bifunctional, performing the aldol condensation and the dehydration in acidic or basic media and the hydrogenation in the presence of a metal.

The catalytic system comprises an acidic or basic solid compound as first component. This compound may be a solid of the zeolite, clay, ceramic, resin or mineral type or any other acidic or basic solid support. By way of acidic solid support, mention may in particular be made of sulphonic acid resins, carboxylic resins, phosphoric resins, inorganic oxides such as sulphated zirconias, acidic clays such as montmorillonites and zeolites, for instance H-ZSM5 and H-Y. By way of basic solid support, mention may be made of compounds bearing, at their surface, hydroxide functions or amine functions, carbonates, metal oxides such as lanthanum phosphates or oxides, basic clays such as layered double hydroxides (LDHs).

The catalytic system comprises, as second component, a metal compound, in particular based on Cr, Co, Ni, Cu, Rh, Pd, Ir, Pt and/or Au. Ni, Pd, Rh and Ir are in particular preferred. This compound may be the metal as such or a metal in hydroxide, oxide or salt form. The metal is preferably in the reduced state for its activity during the hydrogenation.

The metal compound may in particular be used in proportions of between 0.001% and 30% by weight, more preferably between 0.01% and 10% by weight, relative to the weight of the acidic or basic solid compound.

According to a preferred subject of the invention, the catalytic system comprises an acidic or basic solid compound on which the metal compound described above is surface-supported.

Mention may, for example, be made of the Amberlyst® CH28 catalysts from the company Rohm & Haas.

It is also possible for the catalytic system to comprise an acidic or basic solid compound and a metal compound supported on a solid that is inert with respect to the reaction.

The amount of catalytic system can range between 0.01% and 60% by weight, relative to the weight of aldehyde, preferably between 0.1% and 20% by weight, more preferably between 1% and 10% by weight.

Preferably, the reaction is carried out at a temperature of between 10° C. and 200° C., more preferably between 30° C. and 150° C., even more preferably between 80° C. and 120° C.

Preferably, the reaction is carried out at a pressure of between 1 and 100 bar, more preferably between 3 and 25 bar, even more preferably between 8 and 15 bar.

Preferably, the first step is carried out at a temperature of between 10° C. and 200° C. and a pressure of between 1 and 100 bar, more preferably at a temperature of between 30° C. and 150° C. and a pressure of between 3 and 25 bar, even more preferably at a temperature of between 80° C. and 120° C. and a pressure of between 8 and 15 bar.

Such a pressure can be obtained by adding to the reactor a supply of pure hydrogen or of a mixture of hydrogen and an inert gas, for instance nitrogen or argon. The partial hydrogen pressure can be maintained by flushing the gaseous headspace, with the hydrogen content being controlled.

The aldehydes may in particular be acetaldehyde, butyraldehyde or propionaldehyde. One or more aldehydes of different natures may be used.

Preferably, the medium of the reaction comprises, in addition to the catalytic system, only aldehydes. The medium preferably does not comprise solvent and/or compounds capable of reacting during the condensation reaction, such as compounds of alcohol type, for example.

The process according to the invention can be carried out continuously or batchwise, preferably in the liquid phase. The residence time of the first step may in particular be from 5 to 300 minutes.

The reaction of the first step can be carried out in a reactor of any type, in particular in a reaction tube mounted vertically. Several reactors carrying out the process of the first step can be placed in series.

The catalyst can be placed on a fixed bed or else be placed in suspension with agitation in the reactor.

It is possible to carry out one or more steps of purification, in particular by distillation, of the product obtained after the first step, for example in order to recover the reactants.

### Second Step

The second step of the process comprises hydrogenation of the condensed aldehyde, producing alcohol. Thus, the condensed aldehyde is reacted with hydrogen in the gas or liquid phase in the presence of a hydrogenation catalyst, in particular under given temperature and pressure conditions.

The hydrogenation catalyst may be made up of metal or of supported metal containing Cr, Co, Ni, Cu, Ru, Rh, Pd, Ir, Pt or Au, or compounds or mixtures thereof, in particular in concentrations between 0.01% and 30% by weight, preferably between 0.1% and 20%, more preferably between 1% and 10%, relative to the total weight of the catalyst.

Supported or unsupported Raney nickel, Cu/CuO on silica, supported platinum or supported ruthenium may, for example, be used as hydrogenation catalyst.

In this reaction, the temperature can be between 10° C. and 300° C., preferably between 80° C. and 140° C.; and the pressure between 0.1 and 300 bar, preferably between 1 and 100 bar.

Preferably, the process for obtaining alcohol in question envisages the hydrogenation of the butyraldehyde obtained during the aldol condensation of two molecules of acetaldehyde, producing 1-butanol.

In the process for obtaining alcohol in question which envisages catalysts used in the aldol condensation of two molecules of aldehyde and in the hydrogenation of the product of said condensation, said catalysts can correspond to an identical or similar material.

It is possible to carry out one or more steps of purification, in particular by distillation, of the product obtained after the second step.

The process according to the invention makes it possible in particular to obtain 1-butanol from acetaldehyde, 2-ethylhexanol from butyraldehyde or 2-methylpentanal from propionaldehyde.

The two steps described above are preferably carried out in different reactors, in particular arranged one following the other.

The subject of the present invention is also the product that can be obtained by means of the aldol condensation of two molecules of aldehyde as described above.

The subject of the present invention is also the alcohol that can be obtained on the basis of the hydrogenation of the product obtained from the aldol condensation of two molecules of aldehyde, as described above.

The examples of said process, given below, will explain the invention more specifically, purely by way of nonlimiting illustration of the subject or the scope thereof.

### EXPERIMENTAL SECTION

### Example 1

### Batchwise Step 1 Reaction

1.5 g of acidic solid catalyst based on sulphonic resin comprising palladium, and 220 g of acetaldehyde in the liquid state, are charged to a 400 mL stirred reactor. Next, the equipment is closed, pressurized with hydrogen, and heated to a temperature of 100° C. and 12 bar. The conversion reaches up to 32.0% and the butyraldehyde selectivity up to 61.0% in 120 minutes.

### Example 2

### Continuous Step 1 Reaction

1.5 g of acidic solid catalyst based on sulphonic resin comprising palladium are charged to a 400 mL stirred reactor. Next, the equipment is closed and supplied with acetaldehyde in the liquid state (200 g/hour), pressurized with hydrogen, and heated to a temperature of 100° C. and a pressure of 12 bar. The conversion reaches up to 60.0% and the butyraldehyde selectivity up to 90.0%.

### Example 3

### Batchwise Step 2 Reaction

100 g of butyraldehyde and 10 g of Raney nickel catalyst are charged to a 150 mL reactor. Next, the equipment is closed, pressurized with hydrogen, and heated to a temperature of 100° C. and a pressure of 20 bar. A conversion of 99.98% and a 1-butanol selectivity of 98.92% are obtained after 30 minutes.

1-16. (canceled)

17. A process for the condensation of two molecules of aldehyde in the presence of a catalytic system comprising an acidic or basic solid compound and a metal compound, at a temperature ranging from 10° C. to 300° C. and at a pressure ranging from 0.01 to 200 bar, in the presence of hydrogen.

18. The process as defined by claim 17, wherein the acidic or basic solid compound comprises a zeolite, clay, ceramic, resin or mineral.

19. The process as defined by claim 17, the catalytic system comprising an acidic solid compound selected from the group consisting of sulfonic acid resins, carboxylic resins, phosphoric resins, inorganic oxides, sulfated zirconias, acidic clays, montmorillonites, zeolites, H-ZSM5 and H-Y.

20. The process as defined by claim 17, the catalytic system comprising a basic solid compound selected from the group consisting of compounds bearing, at their surface, hydroxide functions or amine functions, carbonates, metal oxides, lanthanum phosphates or oxides, basic clays, and layered double hydroxides (LDHs).

21. The process as defined by claim 17, wherein the metal compound is based on Cr, Co, Ni, Cu, Rh, Pd, Ir, Pt and/or Au.

22. The process as defined by claim 17, wherein the metal compound comprises a metal in the reduced state.

23. The process as defined by claim 17, wherein the metal compound is present in proportions ranging from 0.001% to 30% by weight, relative to the weight of the acidic or basic solid compound.

24. The process as defined by claim 17, wherein the catalytic system comprises an acidic or basic solid compound on which the metal compound is surface-supported.

25. The process as defined by claim 17, wherein the amount of catalytic system ranges from 0.01% to 60% by weight, relative to the weight of aldehyde.

26. The process as defined by claim 17, carried out at a temperature ranging from 30° C. to 150° C.

27. The process as defined by claim 17, carried out at a pressure ranging from 3 to 25 bar.

28. The process as defined by claim 17, wherein the aldehyde is selected from the group consisting of acetaldehyde, butyraldehyde and propionaldehyde.

29. A process for the preparation of an alcohol, comprising first condensing two molecules of aldehyde in the presence of a catalytic system which comprises an acidic or basic solid compound and of a metal compound, at a temperature ranging from 10° C. to 300° C. and at a pressure ranging from 0.01 to 200 bar, in the presence of hydrogen, and then reacting such condensed aldehyde with hydrogen in the gaseous or liquid phase, in the presence of a hydrogenation catalyst.

30. The process as defined by claim 29, for the preparation of 1-butanol.

31. A product produced by aldol condensation of two molecules of an aldehyde as defined by claim 17.

32. An alcohol produced by hydrogenation of the product of an aldol condensation of two aldehyde molecules as defined by claim 17.
